(19) United States
(12) Patent Application Publication
(10) Pub. No.: US 2014/0121284 A1
(43) Pub. Date: May 1, 2014

(54) Stable Pharmaceutical Composition

(71) Applicants: Fang-Hsuing Hsiao, Tainan City (TW); Chia-Sheng Cheng, Tainan City (TW)

(72) Inventors: Fang-Hsuing Hsiao, Tainan City (TW); Chia-Sheng Cheng, Tainan City (TW)

(73) Assignee: Standard Chem. & Pharm. Co., Ltd., Tainan City (TW)

(21) Appl. No.: 13/661,109

(22) Filed: Oct. 26, 2012

(51) Int. Cl. A61K 31/135; A61P 25/24; A61P 25/16; A61P 25/00

(52) U.S. Cl. 514/657

(57) ABSTRACT

Provided is a stable pharmaceutical composition having as an active ingredient a therapeutically effective amount of R(+)-N-propargyl-1-aminoindan or a pharmaceutically acceptable salt thereof, an absorption modulator, and a diluent, wherein the absorption modulator is glycerin, and the diluent is anhydrous dibasic calcium phosphate (CaHPO₄) or microcrystalline cellulose. The stable pharmaceutical composition in accordance with the present invention provides extremely low amount of R(+)-N-propargyl-1-aminoindan and could significantly reduce cost. Moreover, the anhydrous dibasic calcium phosphate (CaHPO₄) used as the diluent could enhance fluidity and improve ingredients uniformity in powder molding process. The glycerin used as the absorption modulator could increase the absorption of active ingredient such as R(+)-N-propargyl-1-aminoindan.

### BACKGROUND OF THE INVENTION

1. Field of the Invention

The present invention relates to a formulation of rasagiline and pharmaceutically acceptable salt thereof, and more particularly to a stable pharmaceutical composition of rasagiline.

2. Description of the Prior Arts

Rasagiline is the R(+) enantiomer of N-propargyl-1-aminoindan, which is a selective irreversible inhibitor of the B-form of the enzyme monoamine oxidase (MAO-B), currently being adopted as the mesylate salt in treatment for certain neurological disorders such as Parkinson's disease, memory disorder, dementia, depression, hyperactive syndrome, head trauma injury or withdrawal symptoms.

U.S. Pat. No. 6,126,968 discloses a pharmaceutical composition comprising an effective amount of racemic S(−) or R(+)-N-propargyl-1-aminoindan, in particular solid dosage forms containing rasagiline as active ingredient, and a pharmaceutically acceptable salt thereof, at least one sugar alcohol such as pentahydric and hexahydric alcohols. However, the mass of active ingredient R(+)-N-propargyl-1-aminoindan in said pharmaceutical composition remains high, which results in high cost of the pharmaceutical composition and unavailability of lower dosage of N-propargyl-1-aminoindan. Moreover, sugar alcohol such as mannitol, xylitol or sorbitol of said pharmaceutical composition is water-soluble, and it would aggregate to form agglomerates in wet granulation process to limit its manufacturing process.

To overcome the shortcomings of instability and high cost, a solution is reducing the content of the active ingredient required in the effective dosage, and admixing the active ingredient with a relatively large amount of excipients for reducing dose of the active ingredient in each tablet. While adding a relatively large amount of excipients, it is difficult to obtain a homogenous distribution of the drug substance in a tablet blend. If a tablet blend with an insufficient distribution of the drug substance is used in tablet manufacture, the tablets so produced lack content uniformity and do not posses acceptable drug content. Poor content uniformity has been shown to cause a marked decrease in bioavailability.

### SUMMARY OF THE INVENTION

Given the aforesaid drawbacks of the prior art such as R(+)-N-propargyl-1-aminoindan as active ingredient being expensive and production of high dosage of the active ingredient according the conventional techniques usually has poor content uniformity, the present invention provides a stable pharmaceutical composition comprising an extremely low amount of R(+)-N-propargyl-1-aminoindan, which can significantly reduce cost, and obviate poor content uniformity and instability by the incorporation of certain diluents with good flow properties.

To achieve the above objective, the stable pharmaceutical composition comprising as an active ingredient a therapeutically effective amount of R(+)-N-propargyl-1-aminoindan or a pharmaceutically acceptable salt thereof; an absorption modulator, and a diluent, wherein the absorption modulator is glycerin, and the diluent is anhydrous dibasic calcium phosphate (CaHPO₄) or microcrystalline cellulose.

Preferably, the therapeutically effective amount of the R(+)-N-propargyl-1-aminoindan ranges from 0.5% to 1% by weight of the total composition.

According to the present invention, use of anhydrous dibasic calcium phosphate (CaHPO₄) in pharmaceutical products contributes to compaction properties and good flow properties of the coarse-grade material.

Preferably, the amount of said anhydrous dibasic calcium phosphate (CaHPO₄) ranges from 50% to 80% by weight of the total composition.

Preferably, the amount of said glycerin ranges from 0% to 5% by weight of the total composition.

Preferably, the amount of microcrystalline cellulose is 0% to 38.8% by weight of the total composition.

In another preferred embodiment, the stable pharmaceutical composition further comprises an extra-granular ingredient, wherein the extra-granular ingredient is selected from the group consisting of corn starch, pregelatinized starch, colloidal silicon dioxide, talc and stearic acid.

According to the present invention, the term “pregelatinized starch”, as used herein, refers to a modified starch used in oral capsule and tablet formulations as a binder, diluent and disintegrant. In comparison to starch, partially pregelatinized starch may be produced with enhanced flow and compression characteristics such that the pregelatinized material may be used as a tablet binder in dry-compression or direct compression processes. In such processes, pregelatinized starch is self-lubricating.

Preferably, the amount of the corn starch ranges from 0% to 20% by weight of the total composition.

Preferably, the amount of the pregelatinized starch ranges from 0% to 20% by weight of the total composition.

According to the present invention, the term “colloidal silicon dioxide” as used herein, refers to a classical solid oral dosage form lubricant providing flow of granules and release of the compressed tablets from the punch and die metallic surfaces which are normally contacted during tableting compression process.

Preferably, the colloidal silicon dioxide is used as glidant, wherein the amount of colloidal silicon dioxide ranges from 0.25% to 1% by weight of the total composition.

Preferably, the talc is used as glidant and lubricant, wherein the amount of talc ranges from 0.5% to 5.0% by weight of the total composition.

According to the present invention, the term “stearic acid” as used herein, refers to oral formulations such as a tablet and capsule lubricant. It may also be used as a binder or in combination with shellac as a tablet coating. It has also been suggested that stearic acid may be used in enteric tablet coatings and as a sustained-release drug carrier.

Preferably, the amount of the stearic acid ranges from 0.5% to 3.0% by weight of the total composition.

In another preferred embodiment, the stable pharmaceutical composition further comprises a citric acid.

Preferably, the amount of the citric acid ranges from 0% to 3% by weight of the total composition.

The stable pharmaceutical composition in accordance with the present invention provides extremely low amount of R(+)-N-propargyl-1-aminoindan and therefore could significantly reduce cost. Moreover, the anhydrous dibasic calcium phosphate (CaHPO₄) used as the diluent can enhance fluidity and improve ingredients uniformity in powder moulding process. Besides, the glycerin used as the absorption modulator can increase the absorption of active ingredient such as R(+)-N-propargyl-1-aminoindan and achieve good bioequivalence.

Other objectives, advantages and novel features of the invention will become more apparent from the following detailed description when taken in conjunction with the accompanying examples.

### DETAILED DESCRIPTION OF THE PREFERRED EMBODIMENTS

### Example 1

Intra-granular ingredients as shown in Table 1 were sifted through a #30 mesh sieve and blended together for 10 minutes in a top spray fluidized bed processor or rapid mixer granulator to form a mixture. Then, 1.56 g rasagiline mesylate was dissolved in water to form a binder solution. Next, the mixture was granulated by spraying with the binder solution in the fluidized bed processor or rapid mixer granulator, and then the granules were dried. Then, the dried granules were sifted through a #40 mesh sieve to form sifted-dried granules. The extra-granular ingredients as shown in Table 1 were sifted through a #60 mesh sieve and blended with the sifted-dried granules to form a composition. Finally, the composition was compressed into tablets.

TABLE 1

| | | |
|---|---|---|
| Ingredient | Percentage (%) | mg |

Intra-granular

| | | |
|---|---|---|
| anhydrous dibasic calcium | 77.46 | 160 |
| phosphate (CaHPO₄) | | |

Binder solution

| | | |
|---|---|---|
| rasagiline mesylate | 0.76 | 1.56 |
| purified water* | — | q.s |

Extra-granular

| | | |
|---|---|---|
| corn starch | 9.68 | 20 |
| pregelatinized starch | 9.68 | 20 |
| colloidal silicon dioxide | 0.48 | 1 |
| Talc | 0.97 | 2 |
| stearic acid | 0.97 | 2 |
| Total | 100.00 | 206.56 |

*Evaporates during processing.

### Example 2

Intra-granular ingredients as shown in Table 2 were sifted through a #40 mesh sieve and mixed to form a mixture. Then, 1.56 mg rasagiline mesylate was dissolved in water to form a binder solution. Next, the mixture was granulated by spraying with the binder solution in the fluidized bed processor or rapid mixer granulator, and the granules were dried at 60° C. Then, the dried granules and 20 mg pre-gelatinized starch of the extra-granular ingredients as shown in Table 2 were respectively sifted through a #40 mesh sieve to form sifted-dried granules and sifted-pregelatinized starch. The sifted-dried granules and sifted-pregelatinized starch were blended to form a complex. 1 mg colloidal silicon dioxide, 2 mg talc, and 2 mg stearic acid were sifted through a #60 mesh sieve to form a compound. Finally, the complex and the compound were blended to form a composition and compressed into tablets.

TABLE 2

| | | |
|---|---|---|
| Ingredient | Percentage (%) | mg |

Intra-granular

| | | |
|---|---|---|
| microcrystalline cellulose | 38.83 | 80 |
| (MCC) | | |
| corn starch | 33.71 | 69.44 |
| pregelatinized starch | 14.56 | 30 |

Binder solution

| | | |
|---|---|---|
| rsasagiline mesylate | 0.76 | 1.56 |
| purified water* | — | q.s |

Extra-granular

| | | |
|---|---|---|
| pregelatinized starch | 9.71 | 20 |
| colloidal silicon dioxide | 0.49 | 1 |
| Talc | 0.97 | 2 |
| stearic acid | 0.97 | 2 |
| Total | 100.00 | 206 |

*Evaporates during processing.

### Example 3

Intra-granular ingredients as shown in Table 3 were sifted through a #40 mesh sieve and blended together for 10 minutes in a top spray fluidized bed processor to form a mixture. Then, 1.56 mg rasagiline mesylate and 6.18 mg citric acid were dissolved in water to form a binder solution. Next, the mixture was granulated by spraying with the binder solution in the fluidized bed processor or rapid mixer granulator, and then the granules were dried. Then, the dried granules were sifted through a #40 mesh sieve to form sifted-dried granules. 20 mg pregelatinized starch of the extra-granular ingredients as shown in Table 3 was sifted through a #40 mesh sieve and blended with the sifted-dried granules to form a complex. 1 mg colloidal silicon dioxide, 2 mg talc, and 2 mg stearic acid were sifted through a #60 mesh sieve to form a compound. Finally, said complex and the compound were blended to form a composition and compressed into tablets.

TABLE 3

| | | |
|---|---|---|
| Ingredient | Percentage (%) | mg |

Intra-granular

| | | |
|---|---|---|
| microcrystalline cellulose | 74.46 | 153.26 |
| (MCC) | | |
| corn starch | 9.68 | 20 |

Binder solution

| | | |
|---|---|---|
| rasagiline mesylate | 0.76 | 1.56 |
| citric acid | 3.00 | 6.18 |
| purified water* | — | q.s |

Extra-granular

| | | |
|---|---|---|
| pregelatinized starch | 9.68 | 20 |
| colloidal silicon dioxide | 0.48 | 1 |
| Talc | 0.97 | 2 |
| stearic acid | 0.97 | 2 |
| Total | 100.00 | 206 |

*Evaporates during processing.

### Example 4

1.56 mg rasagiline mesylate, 20 mg corn starch, and 20 mg pregelatinized starch as shown in Table 4 were mixed to form a homogeneous mixture and the homogeneous mixture was sifted through a #120 mesh sieve. Next, 159.44 mg anhydrous dibasic calcium phosphate and 1 mg colloidal silicon dioxide were sifted through a #40 mesh sieve and added to the homogeneous mixture to form a mixture. Then, 2 mg talc and 2 mg stearic acid were sifted through a #60 mesh sieve and blended with said mixture to form a composition. Finally, said composition was compressed into tablets.

TABLE 4

| Ingredient | Percentage (%) | mg |
|---|---|---|
| rasagiline mesylate | 0.76 | 1.56 |
| anhydrous dibasic calcium | 77.40 | 159.44 |
| phosphate (CaHPO₄) | | |
| corn starch | 9.71 | 20 |
| pregelatinized starch | 9.71 | 20 |
| colloidal silicon dioxide | 0.48 | 1 |
| Talc | 0.97 | 2 |
| stearic acid | 0.97 | 2 |
| Total | 100.00 | 206 |

### Example 5

Intra-granular ingredients as shown in Table 5 were sifted through a #30 mesh sieve and blended together for 10 minutes in a top spray fluidized bed processor to form a mixture. Then, 1.56 mg rasagiline mesylate was dissolved in water to form a binder solution. Next, the mixture was granulated by spraying with the binder solution in the fluidized bed processor or rapid mixer granulator, and then the granules were dried. Then, the dried granules were sifted through a #40 mesh sieve to form sifted-dried granules. The extra-granular ingredients as shown in Table 7 were sifted through a #60 mesh sieve and blended with the sifted-dried granules to form a composition. Finally, the composition was compressed into tablets.

TABLE 5

| | | |
|---|---|---|
| Ingredient | Percentage (%) | mg |

Intra-granular

| | | |
|---|---|---|
| microcrydtalline cellulose | 38.73 | 80 |
| M101 | | |

Binder solution

| | | |
|---|---|---|
| rasagiline Mesylate | 0.76 | 1.56 |
| purified water* | — | q.s |

Extra-granular

| | | |
|---|---|---|
| corn starch | 9.68 | 20 |
| pregelatinized starch | 48.41 | 100 |
| colloidal silicon dioxide | 0.48 | 1 |
| talc | 0.97 | 2 |
| stearic acid | 0.97 | 2 |
| Total | 100.00 | 206.56 |

*Evaporates during processing.

### Example 6

1.56 mg rasagiline mesylate and 10.3 mg glycerin as shown in Table 6 were dissolved in water to form a mixture. 149.14 mg anhydrous dibasic calcium phosphate and said mixture were granulated by spraying in the fluidized bed processor or rapid mixer granulator, and then the granules were dried. Then, the dried granules were sifted through a #40 mesh sieve to form sifted-dried granules. 20 mg pregelatinized starch of the extra-granular ingredients as shown in Table 5 was sifted through a #40 mesh sieve and blended with the sifted-dried granules to form a complex. 1 mg colloidal silicon dioxide, 2 mg talc, and 2 mg stearic acid were sifted through a #60 mesh sieve to form a compound. Finally, the complex and the compound were blended to form a composition and compressed into tablets.

TABLE 6

| | | |
|---|---|---|
| Ingredient | Percentage (%) | mg |

Intra-granular

| | | |
|---|---|---|
| anhydrous dibasic calcium | 72.40 | 149.14 |
| phosphate (CaHPO₄) | | |

Binder solution

| | | |
|---|---|---|
| rasagiline mesylate | 0.76 | 1.56 |
| Glycerin | 5.00 | 10.3 |
| purified water* | — | q.s |

Extra-granular

| | | |
|---|---|---|
| corn starch | 9.71 | 20 |
| pregelatinized starch | 9.71 | 20 |
| colloidal silicon dioxide | 0.48 | 1 |
| Talc | 0.97 | 2 |
| stearic acid | 0.97 | 2 |
| Total | 100.00 | 206 |

*Evaporates during processing.

### Example 7

1.56 mg rasagiline mesylate and 4.12 mg glycerin as shown in Table 7 were dissolved in water to form a mixture. 155.32 mg anhydrous dibasic calcium phosphate and said mixture were granulated by spraying in the fluidized bed processor or rapid mixer granulator, and then the granules were dried. Then, the dried granules were sifted through a #40 mesh sieve to form sifted-dried granules. The 20 mg pregelatinized starch of the extra-granular ingredients as shown in Table 7 was sifted through a #40 mesh sieve and blended with the sifted-dried granules to form a complex. 1 mg colloidal silicon dioxide, 2 mg talc, and 2 mg stearic acid were sifted through a #60 mesh sieve to form a compound. Finally, the complex and the compound were blended to form a composition and compressed into tablets.

TABLE 7

| | | |
|---|---|---|
| Ingredient | Percentage (%) | mg |

Intra-granular

| | | |
|---|---|---|
| anhydrous dibasic calcium | 75.40 | 155.32 |
| phosphate (CaHPO4) | | |

Binder solution

| | | |
|---|---|---|
| rasagiline mesylate | 0.76 | 1.56 |
| Glycerin | 2.00 | 4.12 |
| purified water* | — | q.s |

Extra-granular

| | | |
|---|---|---|
| corn starch | 9.71 | 20 |
| pregelatinized starch | 9.71 | 20 |
| colloidal silicon dioxide | 0.48 | 1 |
| Talc | 0.97 | 2 |
| stearic acid | 0.97 | 2 |
| Total | 100.00 | 206 |

*Evaporates during processing.

### Example 8

The manufacturing process was same as example 5 but the intra-granular ingredient is starlac, which was comprised of 85% α-lactose monohydrate and 15% maize starch dry matter.

TABLE 8

| | | |
|---|---|---|
| Ingredient | Percentage (%) | mg |

Intra-granular

| | | |
|---|---|---|
| starlac | 77.46 | 160 |

Binder solution

| | | |
|---|---|---|
| rasagiline mesylate | 0.76 | 1.56 |
| purified water* | — | q.s |

Extra-granular

| | | |
|---|---|---|
| corn starch | 9.71 | 20 |
| pregelatinized starch | 9.71 | 20 |
| colloidal silicon dioxide | 0.49 | 1 |
| talc | 0.97 | 2 |
| stearic acid | 0.97 | 2 |
| Total | 100.00 | 206.56 |

*Evaporates during processing.

### Example 9

### Stability Test

Samples of example 1 to example 8 were packaged in closed high density polyethylene (HDPE) containers and stored under the stability testing conditions at 40° C. and 75% relative humidity (RH). The impurity content, the percentage of degradations, was analyzed by high-performance liquid chromatography (HPLC) at indicated intervals (0, 1, 2 and 3 months) and results were shown in Table 9, where values were total drug-related impurities The total impurity contents of the all samples were well below the specification limits of 2.0%, respectively. None of the formulation variables showed a statistically significant impact on drug products.

TABLE 9
Total drug-related impurities

| Time | Sample 1 | Sample 2 | Sample 3 | Sample 4 | Sample 5 | Sample 6 | Sample 7 | Sample 8 |
|---|---|---|---|---|---|---|---|---|
| Initial | 0.20 | 0.28 | 0.82 | 0.39 | 0.09 | 0.36 | 0.24 | 2.18 |
| 1 month | 0.29 | 0.30 | 0.89 | — | 3.94 | 0.37 | 0.46 | 5.25 |
| 2 months | 0.64 | 0.53 | 0.87 | — | — | 0.60 | 0.46 | — |
| 3 months | 0.65 | 0.65 | 1.06 | — | — | — | 0.50 | — |

As shown in Table 9, sample 8 was used as control group. While the anhydrous dibasic calcium phosphate (CaHPO₄) of sample 1, sample 4, sample 6 and example 7 replaced starlac of sample 8, the drug-related impurities of sample 1, sample 4, sample 6 and sample 7 were lower than sample 8 at initial released time and 1 month released time. Moreover, the drug-related impurities of microcrystalline cellulose (MCC) of sample 2 and sample 3 are also lower than sample 8 as control group.

### Example 10

### Bioequivalence (BE) Study

The compositions prepared in example 6 were subjected to bioequivalence studies with an open label, randomized two-treatment, two-period, two-sequence, two-way crossover, single dose and comparative oral bio-availability study i.e. composition of example 6 (1 mg) under fasting conditions was carried out. The Azilect® (Rasagiline tablets 1 mg) obtained from Teva (United States of America) was used as control group. There were sufficient numbers of subjects enrolled to dose 6+2 (stand by) in each group. After an overnight fast of at least 10 hours, dosing was done with 240 ml of water at ambient temperature. In each study period a single dose of Rasagiline tablet (1 mg) was administered to the subjects. Subjects received the test formulation in one study period, and received the reference formulation (Azilect® 1 mg) in the other period. Subjects were instructed not to chew or crush the tablets. A total of 13 samples were taken per period at 0.083, 0.167, 0.25, 0.50, 0.75, 1.0, 2.0, 3.0, 4.0, 6.08.0 and 12.0 hours post dose. The results of the above bioequivalence studies under fasting condition were shown in Table 10.

Employing the estimated plasma concentration time profile of rasagiline, pharmacokinetic parameters like Cₘₐₓ, AUC 0-t, AUC 0-inf were calculated. According to international registered guideline, two formulations are bioequivalent if the relative bioavailability of 90% confidence interval (CI) of the test formulation's lies within the range of 0.8 to 1.25 to the reference formulation, and preferred criterion of a test to reference ratio for Cₘₐₓ is between 0.9 and 1.10. The results of the above bioequivalence studies under fasting condition were shown in Table 10.

TABLE 10
Bioequivalence (BE) study

| | |
|---|---|
| Geometric | 90% Confidence Interval |

| Parameters | mean ratio | Lower bound | Upper bound |
|---|---|---|---|
| Ln(AUC 0-t) | 1.0769 | 88.27 | 131.38 |
| Ln(AUC 0-∞) | 1.0653 | 86.92 | 130.56 |
| Ln(Cmax) | 1.3833 | 62.38 | 306.77 |

According to the present invention, the term “Cₘₐₓ” as used herein refers to the maximum plasma/serum/blood concentration of the drug in pharmacokinetics.

According to the present invention, the term “AUC 0-t” as used herein refers to the plasma/serum/blood concentration-time curve from time zero to time t, where t is the last time point with measurable concentration for individual formulation, and the total area under plasma concentration under concentration time curve were calculated by linear trapezoidal rule.

According to the present invention, the term “AUC 0-inf” as used herein refers to the plasma/serum/blood concentration-time curve from time zero to time infinity, where AUC 0-inf=AUC 0-t+Ct/λz, Ct is the last measurable drug concentration and λz is the terminal or elimination rate constant calculate according to appropriate method.

The compositions prepared in example 7 were subjected to bioequivalence studies with an open label, randomized two-treatment, two-period, two-sequence, two-way crossover, single dose and comparative oral bio-availability study i.e. composition of example 6 (1 mg) under fasting conditions was carried out. The Azilect® (Rasagiline tablets 1 mg) obtained from Teva (United States of America) was used as control group. There were sufficient numbers of subjects enrolled to dose 12+2 (stand by) in each group. After an overnight fast of at least 10 hours, dosing was done with 240 ml of water at ambient temperature. In each study period a single dose of Rasagiline tablet (1 mg) was administered to the subjects. Subjects received the test formulation in one study period and received the reference product (Azilect® 1 mg) in the other period. Subjects were instructed not to chew or crush the tablets. A total of 13 samples were taken per period at 0.083, 0.167, 0.25, 0.50, 0.75, 1.0, 2.0, 3.0, 4.0, 6.0, 8.0 and 12.0 hours post dose.

TABLE 11
Bioequivalence (BE) study

| | |
|---|---|
| Geometric | 90% Confidence Interval |

| Parameters | mean ratio | Lower bound | Upper bound |
|---|---|---|---|
| Ln(AUC 0-t) | 0.9502 | 82.75 | 109.11 |
| Ln(AUC 0-∞) | 0.9530 | 83.12 | 109.26 |
| Ln(Cₘₐₓ) | 0.9894 | 80.03 | 122.32 |

The compositions prepared in example 7 were subjected to bioequivalence studies with an open label, randomized, two-treatment, two-period, two-sequence, two-way crossover, single dose and comparative oral bio-availability study i.e. composition of example 6 (1 mg) under fasting conditions was carried out. The experimental conditions are substantially same as above mentioned, but the testing intervals were varied in that a total of 13 samples were taken per period at 0.167, 0.333, 0.50, 0.75, 1.0, 1.5, 2.0, 2.5, 3.0, 4.0, 6.0, 8.0 and 12.0 hours post dose. The results of the above bioequivalence studies under fasting condition were shown in Table 12.

TABLE 12
Bioequivalence (BE) study

| | |
|---|---|
| Geometric | 90% Confidence Interval |

| Parameters | mean ratio | Lower bound | Upper bound |
|---|---|---|---|
| Ln(AUC 0-t) | 1.0144 | 92.12 | 111.70 |
| Ln(AUC 0-∞) | 1.0174 | 92.59 | 111.79 |
| Ln(Cmax) | 0.9853 | 84.55 | 114.81 |

As shown in Table 11 and Table 12, while the glycerin was adjusted from 5% to 2%, it can be seen that the composition of the present invention is bioequivalent to the commercially available Rasagiline tablet in the United States of America, i.e. Azilect®.

Besides, as shown in Table 10 to Table 12, since Cₘₐₓ ratio was from 1.3833 to 0.9894 while the concentration of the glycerin was adjusted from 5.00% to 2.00%, the formulation of 2.00% glycerin is first priority to do pilot bioequivalence and enhance absorption.

### Example 11

### Uniformity Test

Blend uniformity (BU) is a major concern in the formulation and process development stage, and blend uniformity test has been performed on the samples taken from the final blend in the container. Acceptable criteria of blend sample are within 10.0 percent (absolute) of the mean of the results and percent relative standard deviation (% RSD) no more than 5%, but in general, the blend uniformity percent relative standard deviation was even higher than 6%. The blend uniformity of example 1 was shown in the following Table 13.

TABLE 13
Content uniformity results of rasagiline tablets (1 mg)

| | Sample | | | | |
|---|---|---|---|---|---|
| | 1 | 2 | 3 | 4 | 5 |
| % Assay | 101.6% | 100.8% | 100.6% | 103.0% | 100.4% |
| | Sample | | | | |
| | 6 | 7 | 8 | 9 | 10 |
| % Assay | 100.2% | 101.8% | 100.8% | 101.0% | 100.9% |

Average: 101.1%
STD: 0.83%
% RSD: 0.82%

As shown in Table 13, the percent relative standard deviation (% RSD) was only 0.82%, showing that good blend uniformity and content uniformity can be obtained by mixing anhydrous dibasic calcium phosphate (CaHPO₄).

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

1. A stable pharmaceutical composition in tablet form comprising as an active ingredient a therapeutically effective amount of R(+)-N-propargyl-1-aminoindan or a pharmaceutically acceptable salt thereof, an absorption modulator, and a diluent, wherein the absorption modulator is glycerin, and the diluent is selected from anhydrous dibasic calcium phosphate (CaHPO₄) and microcrystalline cellulose;
wherein the amount of said anhydrous dibasic calcium phosphate (CaHPO₄) ranges from 50% to 80% by weight of said total composition, and the amount of said microcrystalline cellulose ranges from 38% to 75% by weight of said total composition.

2. The stable pharmaceutical composition as claimed in claim 1, wherein the active ingredient with therapeutically effective amount of R(+)-N-propargyl-1-aminoindan ranges from 0.5% to 1% by weight of the total composition.

3. (canceled)

4. The stable pharmaceutical composition as claimed in claim 1, wherein the amount of the glycerin ranges from 0% to 5.0% by weight of the total composition.

5. (canceled)

6. The stable pharmaceutical composition as claimed in claim 1, wherein the stable pharmaceutical composition further comprises an extra-granular ingredient, and the extra-granular ingredient is selected from the group consisting of corn starch, pregelatinized starch, colloidal silicon dioxide, talc and stearic acid.

7. The stable pharmaceutical composition as claimed in claim 6, wherein the amount of the corn starch ranges from 0% to 20% by weight of the total composition.

8. The stable pharmaceutical composition as claimed in claim 6, wherein the amount of the pregelatinized starch ranges from 0% to 20% by weight of the total composition.

9. The stable pharmaceutical composition as claimed in claim 6, wherein the amount of the colloidal silicon dioxide ranges from 0.25% to 1% by weight of the total composition.

10. The stable pharmaceutical composition as claimed in claim 6, wherein the amount of the talc ranges from 0.5% to 5.0% by weight of the total composition.

11. The stable pharmaceutical composition as claimed in claim 6, wherein the amount of the stearic acid ranges from 0.5% to 3.0% by weight of the total composition.

12. The stable pharmaceutical composition as claimed in claim 1, wherein the stable pharmaceutical composition further comprises citric acid.

13. The stable pharmaceutical composition as claimed in claim 12, wherein the amount of the citric acid ranges from 0% to 3% by weight of the total composition.
